(19) United States
(12) Patent Application Publication
(10) Pub. No.: US 2009/0104269 A1
(43) Pub. Date: Apr. 23, 2009

(54) Nanoformulations

(76) Inventors: Brian Graham, Dublin, OH (US); Beverly A. Piatt, Columbus, OH (US); James J. Lind, Lenexa, KS (US); James E. Dvorsky, Norwich Township, OH (US); Mark Bell, Fareham (GB); W.M. Prabodha Wijetunge, Plainsboro, NJ (US); Sreedhara Alavattam, Fremont, CA (US)

Correspondence Address: BATTELLE MEMORIAL INSTITUTE, 505 KING AVENUE, COLUMBUS, OH, 43201-2693, US

(21) Appl. No.: 11/883,494

(22) PCT Filed: Feb. 10, 2006
(86) PCT No.: PCT/US06/04770

§ 371 Date: Nov. 21, 2008

Related U.S. Application Data
(60) Provisional application No. 60/652,058, filed on Feb. 11, 2005.
(60) Provisional application No. 60/672,844, filed on Apr. 20, 2005.
(60) Provisional application No. 60/680,126, filed on May 12, 2005.
(60) Provisional application No. 60/690,047, filed on Jun. 10, 2005.

(51) Int. Cl. A01N 25/12; A61K 9/14

(52) U.S. Cl. 424/489

(57) ABSTRACT

Methods, and the products thereof, for producing nanoparticles that in some embodiments are at least partially encapsulated by an encapsulant; and methods, and products thereof, for producing nanoformulations that are suspensions of nanoparticles in a liquid formulation. Typically the nanoparticles include agrochemicals, pharmaceuticals, catalysts, and other active ingredients.

This application claims the benefit of U.S. provisional Application 60/652,058 filed Feb. 11, 2005; U.S. Provisional Application 60/671,844 filed Apr. 15, 2005; U.S. provisional Application 60/680,126 filed May 12, 2005; and U.S. Provisional Application 60/690,047 filed Jun. 10, 2005.

The disclosure and contents of the four cited provisional applications are incorporated by reference herein.

### FIELD OF THE INVENTION

The present invention discloses the use of spray methods to produce nanoparticles of agrochemicals, pharmaceuticals, or other useful chemicals and harvesting the formed nanoparticles. In various embodiments the nanoparticles may be configured into suspensions, larger particles, fibrils, fibers, or a fibrous mat.

### BACKGROUND OF THE INVENTION

Art related to the present invention includes:

1. U.S. Pat. Nos. 5,992,244; 6,093,557; 6,105,877; 6,252,129; 6,318,640; 6,746,869 and 6,764,720;
2. U.S. published applications 2002/0150669 and 2004/0131673 and PCT applications WO 01/87491 A1 and WO 02/076424 A1; and
3. Journal Articles Chuanglong, H. E. et al.; Recent Development of the Nanocomposites Prepared by Coaxial let Technology; Acta Material Compositae Sinica Vol. 22, No. 6, December 2005, and Loscertales I. G. et al., Science, Vol. 295, Mar. 1, 2002, pp. 1695-1698.

### BRIEF DESCRIPTION OF THE INVENTION

A first broad embodiment of the invention includes a method of manufacturing a nanoparticle composition having the steps of: A. preparing a first liquid formulation by combining an active ingredient and a first solvent; B. preparing a second liquid formulation by combining an encapsulant and a second solvent; C. electrically charging the first and second liquid formulation; and D. co-spraying the first liquid formulation and the second liquid formulation to produce a nanoparticle composition, wherein the nanoparticles are at least partially encapsulated.

Another broad embodiment of the invention includes a method and a composition for a nanoparticle composition having the steps of preparing a first solution by combining an active ingredient and a first solvent, wherein the active ingredient is substantially soluble in said first solvent; preparing a second solution by combining an encapsulant and a second solvent, wherein the encapsulant is substantially soluble in said second solvent; and wherein the encapsulant is selected to be substantially soluble in a third solvent, and the active ingredient is substantially insoluble or only partially soluble in the third solvent; and co-spraying the first solution and the second solution, wherein a set of first nanoparticles are formed from at least the first solution, and wherein at least partial encapsulation of the first set of nanoparticles occurs. Finally, an additional step may be added of mixing the at least partially encapsulated active ingredient with the third solvent so as to dissolve the encapsulant and release the active ingredient as a nanoparticle composition. The method includes electrohydrodynamic spraying, electrostatic spraying. In some applications the may be a second active ingredient. In additional embodiments the nanoparticle comprises excipients for the third solvent to stabilize the nanoparticles and prevent crystal growth and Oswald ripening. In various applications the nanoparticle is an agrochemical, a pharmaceutical, a biologically active material, a chemically active material, or mixtures thereof.

In some embodiments the at least partially encapsulated nanoparticle is exposed to a third liquid that that causes oligomerization or polymerization to take place. In some cases the product is treated with UV light to cause further reaction. In some embodiments the first and second solvents are the same, or the third solvent is the same as the first solvent. In some embodiments, the active ingredient is soluble in the first solvent and the third solvent, and the encapsulant is insoluble the third solvent. In other embodiments, the active ingredient is soluble in a third solvent and the encapsulant is caused to swell by the third solvent. In yet other embodiments, the third solvent differs from first and second solvent, but wherein the encapsulant is substantially soluble in the third solvent.

Nozzles are typically selected from the group of a concentric nozzle, a Siamese nozzle, Gemini nozzle, or a wicking nozzle. In some embodiments, the Siamese nozzle or the Gemini nozzle has tapered ends between substantially as illustrated in FIG. 2B, part B, and FIG. 2D, and wherein the angle θ is between about 10° and about 80°. Typically, an electric field is established between the first and second solutions, between the first and second solutions and ground, between the first and second solutions and a target, or combinations thereof.

A further embodiment includes a method for making a nanosuspension comprising providing nanoparticles at least partially encapsulated with an encapsulant; providing a solvent in which the nanoparticles are substantially insoluble and in which the encapsulant is substantially soluble; and mixing the at least partially encapsulated nanoparticles with the solvent to dissolve the encapsulant and forming a suspension of nanoparticles in the solvent. Typically the at least partially encapsulated nanoparticles have an average particle size below about 1000 nm, or wherein the fibers or fibrils have a diameter of less than about 1000 nm. Typically the solution includes excipients to stabilize the nanoparticles and prevent crystal growth and Oswald ripening.

In a yet further embodiment, a method for making a nanosuspension includes providing nanoparticles at least partially encapsulated with an encapsulant; providing a solvent in which the nanoparticles and the encapsulant is substantially insoluble, but causes the encapsulant to swell; and mixing the at least partially encapsulated nanoparticles with the solvent to cause the encapsulant to swell.

An additional embodiment includes a nanosuspension for agricultural use including a solvent; and nanoparticles suspended in said solvent, wherein the nanoparticles comprise an agrochemical and have an average particle size below about 1000 nm. In some embodiments the particles may have a bimodal distribution where one set has a small average particle size and the second set a larger average particle sized

In one embodiment the suspension includes at least partially encapsulated nanoparticles that are insoluble in the liquid in which they are suspended. However, prior to application the particles and the liquid in which they are suspended are mixed with a liquid in which the encapsulant is substantially or at least partially soluble and the new mixture is applied to plants or other surface. The most preferable solvent for the final mixture is water. Therefore the particles are mixed in a nonaqueous solution for distribution and transport, which solution is then mixed with a larger amount of an aqueous solution immediately prior to application.

In some embodiments, the nanosuspension for administration to a patient includes a solvent; and nanoparticles suspended in said solvent, wherein said nanoparticles comprise a pharmaceutical and are administered in a biologically effective amount. The particles may likewise be in a nonaqueous solution and then mixed with an aqueous solution prior to administration.

An additional embodiment includes a method of treating a plant in need of treatment with an agrochemical by providing a suspension of nanoparticles having an agrochemical as an active ingredient; applying the suspension to a plant in need of treatment with the agrochemical, in an effective amount, with our without first mixing the suspension in an aqueous solution.

A yet further embodiment of the invention includes a method for making nanoparticle composition or suspension by the steps of preparing a liquid formulation by combining an active ingredient and a solvent; and spraying the liquid formulation toward the surface of a liquid, wherein the sprayed formulation produces nanoparticles and the nanoparticles are captured by the liquid without forming a mat and forming a nanoparticle composition or suspension of the active ingredient. Typically, an electrical field is established between the liquid formulation and the surface of the liquid during spraying.

An additional embodiment includes a method for making nanoparticle composition or suspension by preparing a first liquid formulation by combining an active ingredient and a first solvent; preparing a second liquid formulation by combining an encapsulant and a second solvent; and co-spraying the first liquid formulation and the second liquid formulation toward the surface of a liquid, wherein the sprayed formulation and encapsulant produce at least partially encapsulated nanoparticles and the at least partially encapsulated nanoparticles are captured by the liquid without forming a mat, and forming a nanoparticle composition or suspension of the active ingredient. Typically an electrical field is established between the first and second liquid formulations, and the surface of the liquid during spraying. In some embodiments, the surface of the liquid is agitated, such as by mixing.

In some embodiments the invention includes a method of manufacturing a nanoparticle composition by the steps of preparing a first liquid formulation by combining an active ingredient and a first solvent; preparing a second liquid formulation with a second solvent, wherein an optional encapsulant may be added; and co-spraying the first liquid formulation and the second liquid formulation toward an edge to produce a nanoparticle composition at said edge.

The edge may be the circular inner circumference of a collection region and may have a plurality of collection regions are disposed at a movable edge. Typically, moving edges are used and the nanoparticle composition forms in the space between the two edges. In some embodiments, the set of edges comprises two or more movable belts and the nanoparticle composition is formed as the belts move so as to form a continuous or semi-continuous mat between the edges. The spray nozzles are typically movable with respect to the edge. In some other embodiments the edge is formed by a rotating plate having one or more openings that define an edge.

### BRIEF DESCRIPTION OF THE DRAWINGS

FIG. 1 is a schematic diagram that illustrates a typical nanoparticle encapsulation and nanoformulation process according to the invention.

FIGS. 2A through 2H are schematic diagrams that illustrate various nozzle configurations according to the invention.

FIG. 3 illustrates particle size determination using light scattering methods. A sample fibrous mat was generated using a Gemini nozzle and the resultant mat was dissolved in water and particle size was determined as in the aqueous dispersion.

FIG. 4 illustrates several embodiments for collecting nanoparticle product according to the invention.

FIGS. 5A and 5B are schematic diagrams that illustrate apparatus for producing a mat containing nanoparticles according to another aspect of the invention.

FIGS. 6A and 6B are schematic diagrams that illustrate apparatus with a plurality of circular edges for continuous production of materials according another aspect of the invention.

FIGS. 7A and 7B are schematic diagrams that illustrate apparatus having a double belt for continuous production of materials according to yet another aspect of the invention.

FIGS. 8A and 8B are schematic diagrams that illustrate apparatus having a rotating plate or disc with one or more openings that define an edge.

FIG. 9 is a schematic diagram illustrating opposing nozzle configurations according to the invention.

FIG. 10 illustrates apparatus and a method for producing nanoformulations or nanosuspensions according to another aspect of the invention.

FIG. 11 illustrates typical apparatus for spraying with a concentric nozzle using an electric charge or electric field.

FIG. 12 illustrates typical apparatus for spraying with a Siamese type nozzle using an electric charge or electric field.

FIG. 13 illustrates typical apparatus for spraying with Gemini type nozzle using an electric charge or electric field.

### DETAILED DESCRIPTION OF THE INVENTION AND BEST MODE

Bioefficacy of certain active ingredients such as agrochemicals and pharmaceuticals can be improved by decreasing their effective size, typically below about 1 um, more typically below about 800 nm, and most typically below about 500 nm. A liquid formulation of active ingredients, typically in aqueous or non-aqueous liquid formulations, is sprayed and the resulting nanoparticles are harvested. To achieve this, several different approaches to produce nanoparticles are used. In one embodiment a nanoparticle is formed and at least partially encapsulated in an encapsulant (e.g. polymer, excipients, or other materials). In addition, excipients are typically used such as surfactants, viscosity enhancing agents, binders, dispersing agents, crystal granule inhibitors, and the like, and other materials to enhance spray characteristics or intermediate or final material properties. Surfactants, wetting agents, and other amphiphiles lower the interfacial tension between the sprayed liquids and the surface of a target such as a surface, edge, or a particle, and permit the liquids to stay on the target. To achieve this, several different approaches to produce nanoparticles are used. Various spraying techniques known in the art such as electric field spraying (electrohydrodynamic spraying, EHD), or others that depend on a critical orifice such as hydraulic or pneumatic pressure spraying, thermally induced spraying, electrostatic ultrasonic spraying (pressure spraying with added high voltage potential, aerolization methods, spray drying, spray freeze drying and the like may be used. To achieve EHD spraying for the formation of nanoparticles, several different nozzle configurations such as concentric, displaced (here referred to as “Gemini”, or Siamese) may be used. In the case with a Gemini nozzle, bombardment with oppositely charged materials typically includes adjuvants, surfactants, viscosity enhancing agents, binders, dispersing agents, crystal granule inhibitors, and the like. Bombardment with oppositely charged materials helps create at least partially encapsulated particles.

In the case of concentric or Siamese nozzles the creation of a single Taylor cone is facilitated that helps in the production of fully or partially encapsulated particles that are easily harvested. In the case of concentric or Siamese nozzle configurations, that are typically at the same voltage or at slightly different voltages of the same polarity, a single Taylor cone is easily created that helps in the production of the partially or fully encapsulated nanoparticles that are easily harvested. The resulting solid matter can be dissolved in an aqueous media and can be used for different functional applications on different targets (e.g. spraying of plants for agrochemical or similar applications) or animals (delivery of drugs for therapeutic applications).

### DEFINITIONS

Electro spraying as used herein means conventional spraying with an electrical component added.

Nanoformulation as used herein means a formulation containing nanoparticles as an active ingredient.

Nano-agrochemical formulation as used herein means a nanoformulation containing agrochemicals in the form of nanoparticles as an active ingredient such as an herbicide, fungicide, pesticide, fertilizer, or growth promoter.

Electric Field Effect Technology (EFET)—includes electrospraying, electrohydrodynamic spraying (EHD, electric field spraying, electro-spinning, spray technology as exemplified by patents such as those to Coffee U.S. Pat. No. 6,252,129, and the like.

Nano-spray—a spray containing nanoparticles or a spray that produces nanoparticles

Nano-dispersions—a dispersion containing nanoparticles

Nano-suspensions—a suspension containing nanoparticles

Electrohydrodynamic spraying (EHD)—a process of spraying a bulk formulation by using electric forces. An electrical charge is applied to the fluid so that as it exits from the spray site, it forms a cone-jet geometry. The jet may break up into aerosol droplet or particles or solidify to form a fiber.

Electric field spraying—electrohydrodynamic spraying whereby the formed jet subsequently breaks up into particles or fibrils (truncated fibers)

Electro-spinning—electrohydrodynamic spraying whereby the formed jet solidifies to a fiber, which may be utilized in this form or collectively combined to form a fibrous mat

Encapsulation as used herein means that the nanoparticle is at least partially or completely encapsulated by an encapsulant. An at least partially encapsulated nanoparticle in some embodiments typically includes a nanoparticle embedded in or adhered to a carrier material. In some embodiments, there is only an active ingredient form without an encapsulant such as when the encapsulant is withheld or when the encapsulant has been stripped off in subsequent processing step.

The liquid formulations useful herein may comprise a solvent, solution, suspension, microsuspension, emulsion, microemulsion, gel or even a melt containing the active component or components. In some embodiments the nanoparticles, nanofibers, or nanofibrils may be in the form of, or within or on, granules, powders, suspensions, solutions, dissolvable films, mats, webs, tablets, or releasable forms particularly releasable dosage forms. Other particular useful forms are concentrates to which a diluting liquid is added prior to use. The product may also be sprayed onto the inner surface of a container to which a liquid is added later prior to use and the nanoparticles, nanofibers, or nanofibrils, are released into the liquid.

Voltages in the range from about 2 kV to about 25 kV are contemplated with different embodiments of the invention that utilize electric field effect technology (EFET).

Agrochemicals useful with the invention are those commonly used in the industry. Representative examples are shown in the following Table 1 which is intended to be exemplary rather than limiting as other materials can be used with the invention.

TABLE 1

| | |
|---|---|
| Type | Representative Examples |

HERBICIDES

| | |
|---|---|
| Amino acids | glyphosate, sulfosate |
| AcetoLactate Synthase inhibitors - | bensulfuron, nicosulfuron |
| Sulfonylurea | |
| ALS inhibitors - Imidazolinone | imazethapyr, imazamox |
| ALS inhibitors - Other ALS | flumetsulam, pyriminobac |
| Triazines | atrazine, simazine |
| Acetamides | acetochlor, metolachlor |
| Dinitroanilines | pendimethalin, trifluralin |
| Aryloxyphenoxypropionates | fluazifop, fenoxaprop |
| Ureas | diuron, isoproturon |
| Carbamates | triallate, phenmedipham |
| Bipyridyls | paraquat, diquat |
| Pyridines | fluroxypyr, trichlopyr |
| Phenoxy acetic acids | 2,4-D, mecoprop |
| Diphenyl Ethers | oxyfluorphen, fomesafen |
| Cyclohexanediones | sethoxydim, tralkoxydim |
| Hydroxybenzonitriles | bromoxynil, ioxynil |
| Pyridazines | chloridazon, norflurazon |
| Other Herbicides | dicamba, bentazone |

INSECTICIDES

| | |
|---|---|
| Organophosphates | chlorpyrifos, monocrotophos |
| Pyrethroids | cypermethrin, deltamethrin |
| Carbamates | aldicarb, methomyl |
| Neonicotinoids | imidacloprid, thiamethoxam |
| Acaricides | clofentezine, fenbutatin oxide |
| Natural Products | Bacillus thuringiensis, Abamectin |
| Benzoylureas | lufenuron, chlorfluazuron |
| Other Insect Growth Regulators | buprofezin, cyromazine |
| Organochlorines | endosulfan, dicofol |
| Other Insecticides | fipronil, chlorfenapyr |

FUNGICIDES

| | |
|---|---|
| Sterol Biosynthesis inhibitors - | epoxiconazole, tebuconazole |
| Triazoles | |
| Sterol Biosynthesis inhibitors - | probenazole, tricylazole |
| Other Azoles | |
| Sterol Biosynthesis inhibitors - | fenarimol, nuarimol |
| Others | |
| Sterol Biosynthesis inhibitors - | dimethomorph, fenpropimorph |
| Morpholines | |
| Multisite - Dithiocarbamates | mancozeb, maneb |
| Multisite - Inorganics | copper fungicides, fentin |
| Multisite - Phthalimide/ | chlorothalonil, captan |
| Phthalonitriles | |
| Multisite - Others | quintozene, dithianon |
| Strobilurins | azoxystrobin, kresoxim methyl |
| Benzimidazoles | carbendazim, benomyl |
| Phenylamides | metalaxyl, oxadixyl |
| Dicarboxamides | iprodione, vinclozolin |
| Carboxamides | carpropamid, carboxin |
| Anilinopyrimidines | cyprodinil, pyrimethanil |

Excipients useful with the invention are those commonly used in the industry.

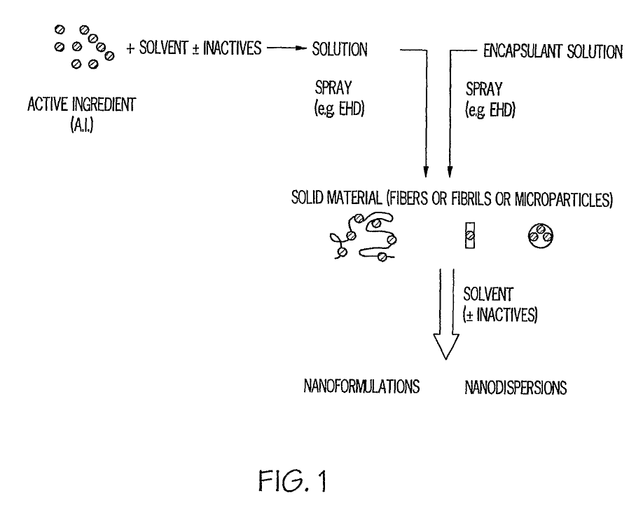

Bioefficacy of certain agrochemical formulations can be improved by decreasing their effective size to below 1 micron. Submicron particles can be generated under certain conditions of flow rate, nozzle geometry, charging potential, and physical properties of formulations. The present invention uses the methods disclosed herein to spray a liquid formulation of agrochemicals (typically non-aqueous) and harvest the formed particles (nanoparticles), or harvesting formed nanoparticles on or in a carrier or in a releasable form. In one embodiment according to the invention, an EHD spray process is used to spray non-aqueous solutions of agrochemical or other materials in combination with and EHD process to generate a carrier/encapsulant to harvest the formed particles (nanoparticles of active ingredient). A simplified representation is shown in the schematic according to FIG. 1. FIG. 1 shows that active ingredient 100 is added to a solvent 110 in which the active ingredient is substantially soluble along with any other desired inactive ingredients 120 to form a liquid formulation or solution 122. An encapsulant liquid formulation 124 may also be prepared and the two may be sprayed 130 simultaneously (e.g. by electrohydrodynamics, EHD) so that the active ingredient forms an active ingredient nanoparticle 140 (as shown) that is at least partially encapsulated by encapsulant. The product of the spraying operation is typically a solid material such as a particulate on a fiber 132, a fiber 134, or a particulate 136 powder or a mat. Typically the powder is a fiber, fibril, nanoparticle, or microparticle (may be solid, semisolid, or a gel) where in some embodiments an active ingredient nanoparticle may be at least partially encapsulated. The product may be made into further end products by dissolving the product in a solvent (e.g. water) to form a liquid formulation having suspended nanoparticles and dissolved encapsulant. The latter end product that contains active ingredient nanoparticles is typically referred to as a nanoformulation or nanodispersion.

Pharmaceutically or biologically active ingredients may be added to the liquid formulation before it is supplied to the spray nozzles. Possible active components are one or more of the following, namely pharmaceutical compounds such as analgesics, antiseptics, antibiotics, bactericides, antifungals, antiparasitics, anti-inflammatory agents, vasodilators (such as minoxidil which is believed to promote wound epithelialization and neovascularization), agents such as proteolytic enzymes for debridement and tissue repair promoting materials such as for example cytokines for stimulating cytokinetic activity to promote essential cell activities, for example to stimulate dendritic growth, growth factors such as fibroblast growth factor (FGF), epithelial growth factor (EGF), transforming growth factor (TGF) that are believed to reduce scarring and others that may be used to promote or otherwise control the sequence of events essential to natural tissue repair, cells, peptides, polypeptides, insulin, immune suppressants or stimulants and vaccines. Other possible active components are DNA or other genetic matter for gene therapy, surface binding or surface recognizing agents such as surface protein A, and biologically active surfactants.

The harvesting of nanoparticles has been challenging (e.g. in a chamber). The present invention discloses several solutions to solve this problem.

A first embodiment of the invention co-sprays a liquid formulation of an active ingredient along with an encapsulant liquid formulation typically containing a polymer (e.g. polyvinylpyrollidone, PVP) liquid formulation using various nozzle configurations. In one aspect the active ingredient, nanoparticles are associated with a fiber, particle or other solid, semisolid, or gel form that will have different properties (quick dissolve, slow dissolve, controlled release etc). Quick dissolve format fibers have been efficiently created by the inventors using an EFET fiber spraying process previously disclosed in patents such as U.S. Pat. No. 6,252,129. A combination of nanoparticles and fibers can typically provide not only better efficacy, but also provide a new delivery mechanism.

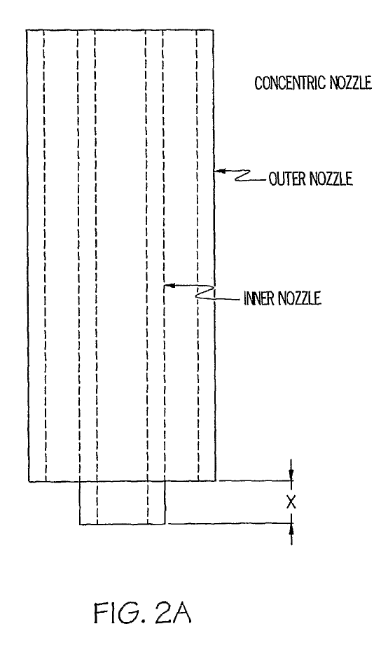

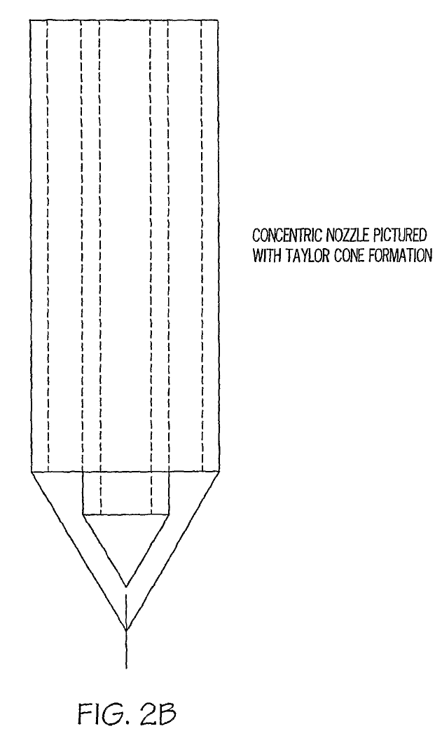

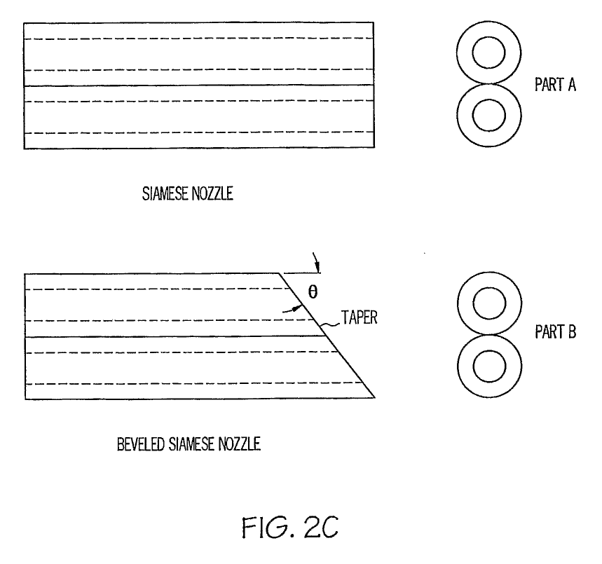

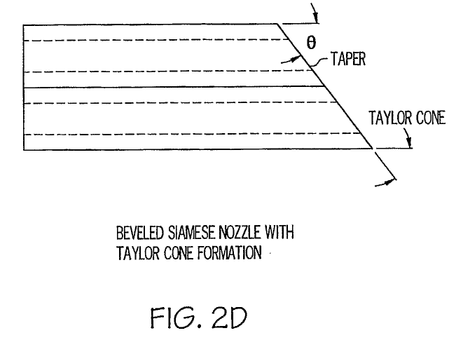

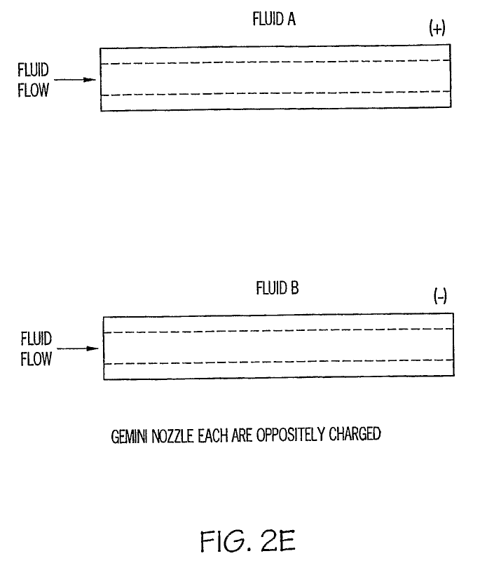

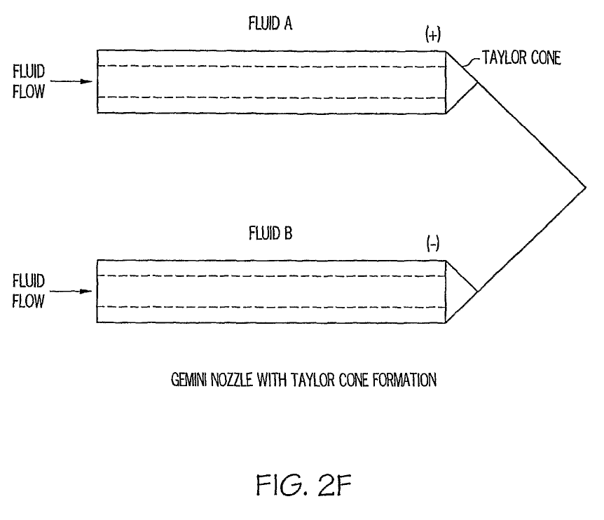

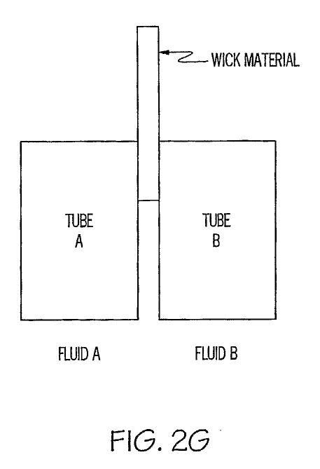

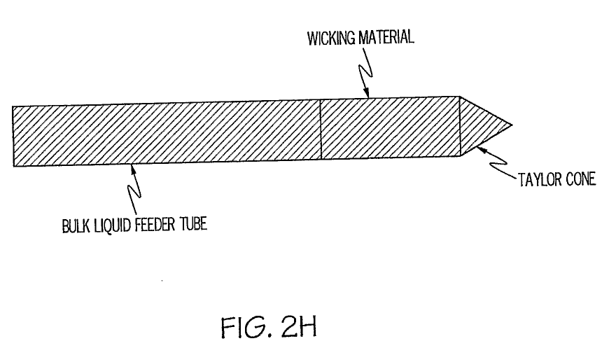

Referring now to FIGS. 2 through 2G, various nozzle configurations were tested to spray or co-spray these formulations and produce sub-micron particles using EFET atomization techniques. For Examples 1A, 2A, and 3A herein surrogate active ingredients (e.g. fluorescein or bifenthrin) were used. These surrogate active ingredients were useful for demonstrating the concept and creating nanosize active ingredient particles. The following nozzles and formulations were used:

(A) Concentric Nozzle: This design is a nozzle inside a nozzle, see FIGS. 2A and 2B. Each of the nozzles are tubes, and the sizes of the tubes were calculated to make sure that the inner tube fit into the outer tube with some clearance space. That clearance space is the fluid path of the outer nozzle, while the inner diameter of the inner tube was the fluid path of the inner nozzle. If one pumping mechanism is used then the inner and outer nozzles should be sized to give approximately the same fluid velocity exiting the tubes. However, in some applications fluid velocities that are not the same are contemplated depending on the particle sizes and fluid properties. Several nozzle size configurations used. An example of one nozzle: the outer nozzle has an internal diameter of 0.050″ and outer diameter of 0.058″. The inner nozzle has an internal diameter of 0.020″ and outer diameter of about 0.028″. The nozzles were charged at the same polarity, positive in this case. In several examples encapsulated nanoparticles in a fiber using EFET were produced. This was accomplished by spraying several concentrations of polyvinylpyrollidone in ethanol in the outer nozzle and a surrogate active ingredient in an acetone solution in the inner nozzle. The resultant sprays were collected on glass slides and viewed under a microscope. Optical pictures of these glass slides indicated that the active ingredient was encapsulated within the fiber. A summary of the tests are described in Table 2. FIG. 2A shows a concentric nozzle 200 for the invention having inner 210 and outer 220 nozzles. The inner nozzle 210 may extend beyond the outer nozzle 220 by a distance X. FIG. 2B shows a concentric nozzle with Taylor cone formation.
(B) Siamese Nozzle: This nozzle configuration includes twin nozzles attached adjacent to one another with the ends normal to the same plane. The nozzles were probe stainless steel point hypodermic needles attached together with a small diameter (about 0.010″) stainless steel wire. The nozzles were charged with same potential, positive in this case. Different fluids were pumped through the nozzles with a single pumping mechanism to produce the same flow rate in each nozzle. Multiple pumping mechanisms could be used if different flow rates per nozzle were required. The surrogate active ingredient (fluorescein) was dissolved in acetone at 2.5 mg/ml and used along with polyvinylpyrollidone in ethanol at about 5.8 mg/ml. However under these circumstances the aerosol produced was inconsistent. The nozzle geometry did not provide a stable base for the Taylor cones to form and remain stationary. In order to provide smooth fluid transition the end of the nozzle where the Taylor cone would form, the ends of the nozzles were beveled or mitered to an angle. The bevel was created by surface treatment of the nozzle at an approximate 45 degree angle to the surface of fine grit sand paper. See FIGS. 2C and 2D. FIG. 2C part A illustrates a Siamese nozzle that is 250 that is joined while FIG. 2C part B shows a Siamese nozzle that has a tapered surface of angle θ. This allowed the fluid to mix as it flowed out of the ends and aerosolize at the outermost tip. The beveled edges were advantageous with this set of fluids to create a Taylor cone that helped in creating an encapsulated end product. Consistent, well established cone-jets and sprays were observed. Several glass slides were placed in the path of the spray and these samples were visualized under the microscope. Details of the experiment are given in Table 2.

A plurality of beveled (above two) may used as for example 3, 4, 5 and so on with each having independent formulations and flow rates.

(C) Gemini Nozzle: The concept of this nozzle was to use two different nozzles separated from each other, one nozzle was positively charged, while the other was negatively charged. When opposing charges are used the resulting aerosol or spray would have enhanced mixing or capture of the formulations after atomization, thus reducing problems related to formulation, solvent and active ingredient compatibility. For the purpose of proof of concept, fluorescein in acetone was used as one formulation and polyvinylpyrollidone in ethanol as the other formulation. The acetone/fluorescein formulation was sprayed at negative voltage, while the polyvinylpyrollidone/ethanol solution was sprayed at positive voltage. Both formulations were sprayed simultaneously and collected on a glass slide where the back of the slide was covered in aluminum foil that was electrically grounded. These slides were viewed under the microscope. The samples were collected for about 20 seconds and a minimum amount of water was added to these, dissolving the resultant sprayed fibers and active ingredient particles. The details of the experiment are provided in Table 2. See FIGS. 2E and 2F.

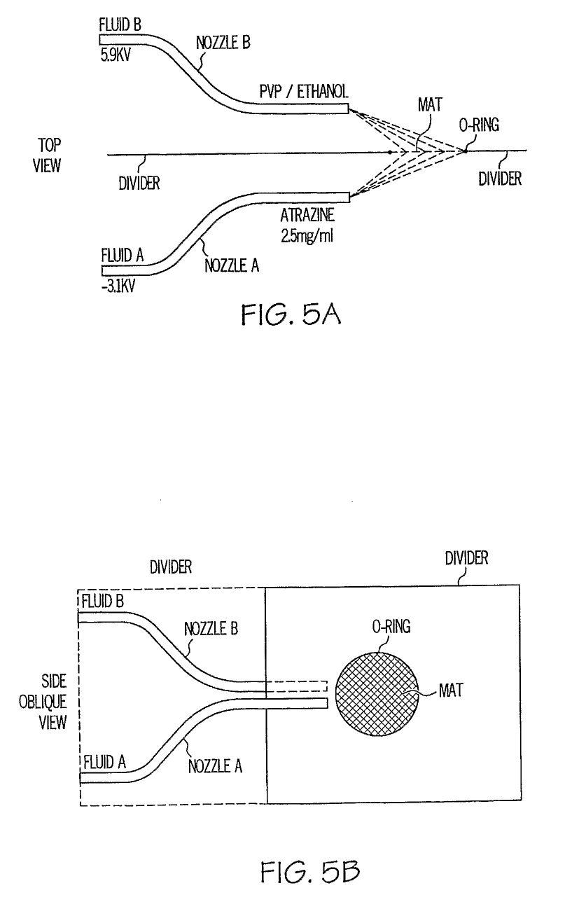

The fibers formed by this method may be collected with an apparatus similar to FIGS. 5A, 5B, 6A, 6B, 7A, and 7B. FIGS. 5A and 5B illustrate a discontinuous method using apparatus 500 with one circular edge (e.g. O-ring) 502. Fluids a and b are co-sprayed from nozzle A 504 and nozzle B 506 respectively. An optional divider 510 may be used to separate sprays 512, 514 until they reach the desired location for combining at 516 where in this case a mat 518 is formed in the space within the O-ring 502.

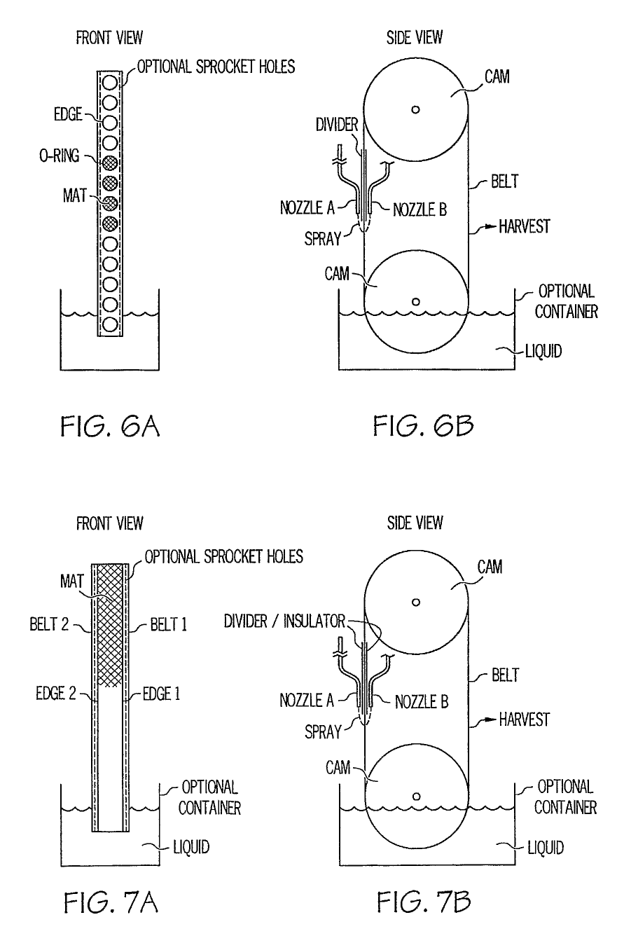

FIGS. 6A and 6B illustrate an apparatus 600 having a continuous belt 602 with circular edges 604 typically formed by an O-ring 606. The belt 602 may have sprocket holes 608 for advancing and keeping the belt 602 aligned and typically runs on a cam 610. The belt 602 typically moves through a bath of liquid 612 for coating the collected material or for collecting the sprayed material. The liquid may be held in optional container 614. Nozzles A and B, 620 and 622 respectively, are used to spray the liquids. An optional divider 630 may be used. A mat 640 typically forms at the edge 604.

FIGS. 7A and 7B illustrate an apparatus having a dual belt 702 703 and double set of edges 704 and 705 within a set of a continuous band. The continuous bands may include a movable set of a plurality of circular edges 701-1, 701-2 or a movable double continuous belt having two edges (Edge 1, Edge 2) with a central opening 701A. The belts 702, 703 may have sprocket holes 708, 709 for advancing and keeping the belts 702, 703 aligned and typically run on a cam 710. The belts 702, 703 typically move through a bath of liquid 712 for coating the collected material or for collecting the sprayed material. The liquid may be held in optional container 714. Nozzles A and B, 720 and 722 respectively, are used to spray the liquids. An optional divider 730 may be used. A mat 740 typically forms between the edges 701-1, 701-2.

D) Polymer wicking nozzle: This embodiment uses a wick typically constructed of a polymer made out of a high surface energy plastic, a wicking material such as a candle wick or a polymer of high surface energy cloth or web, which would provide mixing of the fluids in the wick. The fluid chambers could be separated by space, and the wick would be the joining of the fluid chambers. High voltage would be applied to the fluid chamber, which would provide the aerosolization at the other end of the tip of the wick. It is also possible to generate an electrohyrodynamic spray from one or multiple Taylor cones from various materials which in essence represent a wick. A wick can be defined as a fibrous or spongy material that has intercellular spaces through which the material can migrate or move. The wick can be constructed from several different materials (e.g. polymers, natural fibers). See FIGS. 2G and 2H.

The electrohydrodynamic method for creating the nanoparticles is preferred because the particle sizes produced can be generally produced within a tight distribution of particle sizes. As well, the electrohydrodynamic method may be adjusted to select and produce nanoparticles having a generally pre-determined size. The particle size produced by electrohydrodynamics is such that the solvent for the active ingredient can be evaporated or flashed off quickly. The resulting nanoparticle of active ingredient is then captured by the material produced by the second nozzle in accordance with the invention. It is believed that dilution of the molecular solution of active ingredient in a solvent will affect the size of the resulting nanoparticle of active ingredient produced, and is one factor affecting the desired size of the active ingredient nanoparticle.

Formulation Strategies: The inactive ingredient formulation to be sprayed (e.g. via EFET) in one of the above configurations typically will consist of a solution of one or more of the adjuvant, polymer, surfactant and other inactive ingredients necessary to make an agrochemical formulation. Such chemicals are known in the art and include polyvinylpyrollidone, Morwet™, Silwets™, Pluronic™, polysaccharide gums, polysaccharides, small molecule stabilizers, ionic and non-ionic surfactants and various other inactive ingredients. A typical list of amphiphilic materials useful for the formulations is provided immediately below.

Typically an amphiphilic material can function as a surfactant to reduce the surface and interfacial tension between two immiscible liquids, or as an agricultural adjuvant to enhance the activity of the active ingredient. The amphiphilic material can be selected from cationic surfactants, non-ionic surfactants, quaternary surfactants, amphoteric surfactants, zwitterionic surfactants, and combinations thereof. The cationic surfactant can be an alkylamine having a carbon chain length of between 8 and 18 (including unsaturated carbon chains such as an oleyl group), an alkoxylated amine having between 8 an 18 carbon atoms, such as Ethomeen® S/12 (bis(2-hydroxylethyl)soyaalkylamine), Ethomeen® S/15 (polyoxyethylene (5) soyaalkylamine), and Ethomeen® S/25 (polyoxyethylene (15) soyaalkylamine), which are available from Akzo Nobel Surface Chemistry LLC, Chicago, Ill., and combinations thereof. The non-ionic surfactant can be a polyoxyethylene alcohol such as Brij™ 93 (polyoxyethylene (2) oleyl ether) or Brij™ 97 (polyoxyethylene (10) oleyl ether), a polyoxyethylene sorbitan fatty acid ester such as Tween™ 80 (polyoxyethylene (20) sorbitan monooleate), and combinations thereof, which are available from Uniqema, New Castle, Del., acetylenic and ethoxylated acetylene diol surfactants such as Surfynol™ 104, Surfynol™ 420, Surfynol™ 440, etc., which are available from Air Products and Chemicals, Inc. of Allentown, Pa., and combinations thereof. In addition, the amphiphilic material can be selected from alkylamines, alkylamine ethoxylates, alkylamine propoxylates, alkylamine propoxylates-ethoxylates, fatty alcohol ethoxylates, fatty alcohol propoxylates, fatty alcohol propoxylates-ethoxylates, fatty acid ethoxylates, fatty acid propoxylates, fatty acid propoxylates-ethoxylates, synthetic long-chain alcohol ethoxylates, synthetic long-chain alcohol propoxylates, synthetic long-chain alcohol propoxylates-ethoxylates, synthetic long-chain acid ethoxylates, synthetic long-chain acid propoxylates, synthetic long-chain acid propoxylates-ethoxylates, alkylphenol ethoxylates, alkylphenol propoxylates, alkylphenol propoxylates-ethoxylates, alkylpolyglucosides, sorbitol esters, sorbitan esters, sorbitol ester ethoxylates, sorbitan ester ethoxylates, polyoxypropylene-polyoxyethylene block copolymers (e.g., BASF's “PLURONICS™”), ethylenediamine-polyoxypropylene-polyoxyethylene block copolymers (e.g., BASF's “TETRONICS™”+) and combinations thereof.

The active ingredient formulation can be made to dissolve completely in a solvent of choice, such as chloroform, acetone, dichloromethane, dimethylformamide, dimethylsulfoxide, and the like. Once a molecular solution is achieved, the active ingredient formulation will be co-sprayed with the other formulation using one of the above mentioned nozzle configurations to create a solid dose form. Once the solid composition is prepared using any of the nozzles or concepts given above, the formulation can be dissolved in a solvent (e.g. aqueous media) that will be sprayed on to the required plant material (when active ingredients an agrochemical) or ingested by animals or human (when the active ingredient is a pharmaceutical). The solvent can have one or more surfactants, polymers, dispersants, adjuvants, penetration enhancers, stabilizers and other inactive ingredients in order to prevent agglomeration, crystallization or Oswald ripening. In case of human or animal use, the solid composition can be ingested without adding it into an aqueous media.

One or more active ingredients can be used in the formulation which results from the process of the present invention. The nanoparticles of active ingredients may be created using the electrohydrodynamic process described herein, together from one nozzle configuration of the present invention; or two separate active ingredients may be produced from separate nozzles of the present invention. Further, multiple active ingredients may be used where one active ingredient is produced as a nanoparticle in accordance with the present invention, and the other active ingredient is of a more conventional size and embedded or adhered or encapsulated by a polymer or carrier produced from another electrohydrodynamic nozzle or from other means. Thus, one or more nanoparticles of active ingredient can be produced from a formulation sprayable by electrohydrodynamics, or combined with one or more active ingredients of larger particle size carried by the polymer carrier. An example of the latter case would be a nanoparticle of an agrichemical active ingredient whose efficacy and uptake is enhanced by combination with another active ingredient of conventional size

Three nozzle configurations tested are shown in the FIGS. 2A-2F. Other configurations for spraying two liquids, one liquid and one solid, a liquid and an aerosol etc are also feasible via EFET. The nozzle configurations are not limited to one or two nozzles and can be extended to have an array of nozzles that perform a similar task. FIG. 2G illustrates a nozzle configuration 290 where a wicking material 292 aids in spray formation. FIG. 2H shows another nozzle configuration 296 where a wicking material 297 at the end of a bulk feeder tube 298 forms a Taylor cone 299.

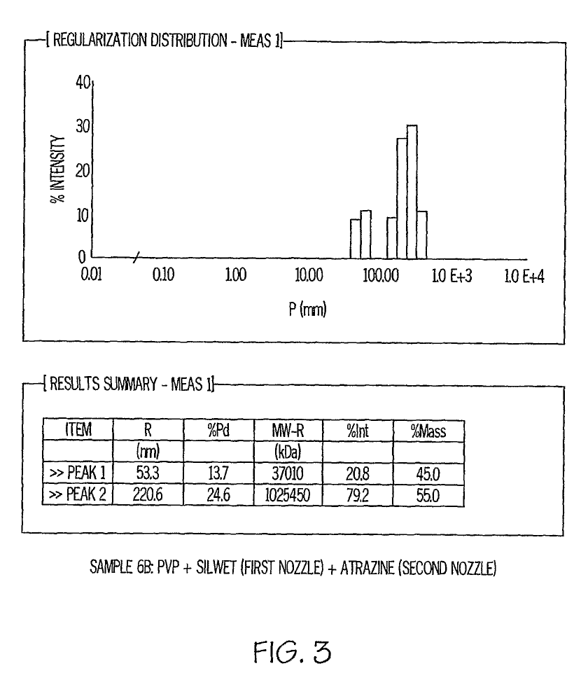

FIG. 3 is a graph showing the particle size distribution of sample 6B dissolved in water and run through a DynaPro™ instrument to measure nanometer particle sizes based on light scattering techniques. Fibers generated using the Gemini nozzle were collected and dissolved in 1.5 mL of water. The water was previously filtered through a 0.02 μm filter. The % intensity (light scattering intensity) and the % mass refer to that of particles of less than 1000 nm size.

As shown in FIG. 3, the formulations can be dissolved in water to get particles in the nano to submicron (250-800 nm) range.

Table 2 summarizes experimental details for typical nanoformulations according to the invention.

TABLE 2
Active Ingredient Cosprayed with Encapsulant

| | | | | | | | Encapsulated |
|---|---|---|---|---|---|---|---|
| | | | | | | | or At least |
| | | | | | | Flow | Partially |
| | | | Nozzle | Voltage 1 | Voltage 2 | Rate | Encapsulated |
| No. | Formulation 1 | Formulation 2 | Configuration | (kV) | (kV) | (uL/min) | Matter |
| 1A | Bifenthrin/ | PVP/Ethanol | Concentric | 7.6 | N/A | 5 | Yes |
| | Acetone | | | 10 | N/A | 20 | |
| | | | | 13.3 | N/A | 10 | |
| 2A | Fluorescein/ | PVP/Ethanol | Siamese | 5.8 | N/A | 5-10 | Yes |
| | Acetone | | | | | | |
| 3A | Fluorescein/ | PVP/Ethanol | Gemini | Positive | Negative | 20 | Yes |
| | Acetone | | | 5.9 | 3.1 | | |

PVP = polyvinylpyrollidone

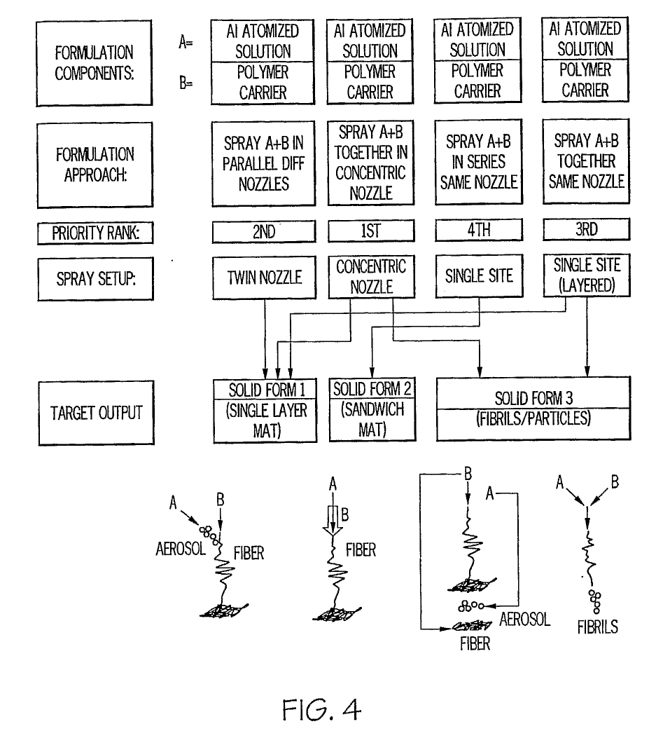

FIG. 4 illustrates several embodiments of the invention. Formulation Components 401, having parts a and b, are listed to the right; Formulation Approaches 411 are listed to the right; Priority Rankings 421 in terms of presently preferred approaches are listed the right; Spray Setup configurations 431 are listed to the right; and Target Outputs 441 are also listed to the right.

The first illustration at the bottom of the page is a single layer mat 461 that incorporates discrete aerosol particles 462 into a fiber 463; the second illustration is for another single layer mat 471 that incorporates fibers 472 with active ingredients (b) 473 into mat 471; the third illustration shows a sandwich mat 481 made from an upper layer of fiber 482 from component b, a lower layer of fiber 484 from component b and a middle layer of particles 483; and the fourth illustration shows fibrils 491 made form components a and b.

The following examples are intended to be illustrative of several aspects of the invention and are not intended to restrict the scope of the invention in any way.

### EXAMPLE 1

Polyvinylpyrollidone formulation: Dissolve 3 g of PVP K30® and 3 g of PVP K90® (both from Sigma, USA) in 25 g of absolute ethanol (200 proof). (K30 and K90 refers to the average viscosities for the respective PVP samples and represents an average molecular weight of 40 kDalton and 360 kDalton respectively) This formulation was stirred at room temperature for about an hour. To 7.5 g of the above formulation, 17.5 g of absolute ethanol was added, mixed well and used for spraying in the nozzle configurations given above. The final concentration of the polyvinylpyrollidone/ethanol solution was about 5.8 wt %.

### EXAMPLE 2

Active Ingredient formulation: The surrogate active ingredient (bifenthrin or fluorescein) was dissolved in acetone at concentrations of 2.5 mg/mL.

One embodiment of this invention includes an active ingredient delivery system to and/or a manufacturing process for active ingredients, specifically active ingredients that are insoluble in water.

This process could be scaled up to a manufacturing process that could represent a novel active ingredient particle size reduction and/or encapsulation process.

This formulation method/process is a novel method to transform a solid active ingredient (particle size greater than 1 micron), that is insoluble in water, into an active ingredient composition (smaller than 1 micron) incorporated in an aqueous-based solution.

An agrochemical or pharmaceutical active ingredient composition is expected to exhibit improved biological performance if it is smaller than 1 micron as opposed to being larger than 1 micron in size.

Additional spray tests were performed for fluorescein and atrazine as active ingredients, with the results shown in the following Table 3.

TABLE 3
Nanoformulations from Fluorescein and Atrazine

| | Formulation 1 | Formulation 2 | | | | Average |
|---|---|---|---|---|---|---|
| | Active Ingredient/ | Encapsulant/ | Surfactant in | | | Particle |
| | First Solvent | Second Solvent | Formulation 2 | Third | Time | diameter |
| No. | (mg/mL) | (mg/mL) | (wt %) | Solvent | (hours) | (nm) |
| 3-1 | Fluorescein/Acetone/2.5 | PVP/Ethanol/5.8 | 0 | water | 0 | 360 |
| 3-2 | Fluorescein/Acetone/2.5 | PVP/Ethanol/5.8 | Silwet L7001 ™/2.5 | water | 0 | 386 |
| 3-3 | Fluorescein/Acetone/2.5 | PVP/Ethanol/5.8 | Pluronic L31 ™/2.5 | water | 0 | 274 |
| 3-5 | Atrazine/Acetone/2.5 | PVP/Ethanol/5.8 | 0 | water | 0 | 302 |
| 3-6 | Atrazine/Acetone/2.5 | PVP/Ethanol/5.8 | Silwet L7001 ™/2.5 | water | 0 | 442 |
| 3-7 | Atrazine/Acetone/2.5 | PVP/Ethanol/5.8 | Pluronic L31 ™/2.5 | water | 0 | 336 |
| 3-11 | Atrazine/Acetone/2.5 | PVP/Ethanol/5.8 | 0 | 0.05 wt % | 0 | 362 |
| | | | | Silwet L7001 ™ | | |
| | | | | in water | | |
| 3-12 | Atrazine/Acetone/2.5 | PVP/Ethanol/5.8 | 0 | 0.05 wt % | 24 | 306 |
| | | | | Silwet L7001 ™ | | |
| | | | | in water | | |
| 3-13 | Atrazine/Acetone/2.5 | PVP/Ethanol/5.8 | 0 | 0.05 wt % | 96 | 314 |
| | | | | Silwet L7001 ™ | | |
| | | | | in water | | |

All experiments were conducted at room temperature about 25° C. The particles were collected under the hood with the hood air blowers off.
The average particle diameter (nm) refers to that of particles below 1000 nm.

The apparatus used for the tests in Table 3 was a two nozzle “Gemini” configuration as shown in FIGS. 5A and 5B. Formulation 1 (Fluid a at nozzle A) was sprayed at −3.1 kV and Formulation 2 (Fluid b, at nozzle B) was sprayed at +5.9 kV. All flow rates were at 20 uL/min. The device has a divider that holds the mat being formed. The divider was a typical clear plastic of high dielectric constant.

For the examples of Table 3, all the product was collected as nanoparticles within fibers. The collected fibers were then dissolved in a third solvent, which was either water or 0.05 wt % Silwet L7001 in water. The time shown in Table 3 is the time delay between the dissolution of the product fibers in the solvent and the time when the particle size was measured.

### EXAMPLE 3-1

### Sample #03-04-05-13 Analysis

A stock solution was prepared from 3.07 g of PVP K30™, 3.06 g of K90™, and 25.30 g of ethanol (200 proof). The stock solution was stirred for two hours at room temperature.

7.5 g of the stock solution was diluted with 17.5 g of ethanol. This preparation was labeled as the working solution. 25 mg of fluorescein was dissolved in acetone to make a 2.5 mg/mL solution by stirring for 10 minutes. The PVP working solution and fluorescein in acetone was co-sprayed using EFET. Fibers were produced on co-spraying and collected on a Teflon™ O-ring. The collected fibers were dissolved in water that had been filtered with a 0.02 um filter prior to adding to the fiber. The final solution was not filtered. Particle size was measured with a Dynapro™ machine and it was assumed that the particles would behave similar to a globular protein. Water was the solvent in the analysis.

### EXAMPLE 3-2

### Sample #03-04-05-21 Analysis

0.25 g of Silwet L7100 was dissolved in 9.76 g of PVP working solution from Example 3-1. This solution and fluorescein in acetone (2.5 mg/mL) were co-sprayed using EFET. Fibers were formed and collected on a Teflon™ O-ring. The fibers collected were dissolved in water that had been filtered with a 0.02 um filter prior to adding the fiber. The final solution was not filtered. Particle size was measured with a Dynapro™ machine and it was assumed that the particles would behave similar to a globular protein. Water was the solvent in the analysis.

### EXAMPLE 3-3

### Sample #03-04-05-31 Analysis

0.26 g of Pluronic L31™ was dissolved in 9.78 g of PVP working solution from Example 3-1. This solution and fluorescein in acetone (2.5 mg/mL) was sprayed using EFET and collected on a Teflon™ O-ring. The fibers collected were dissolved in water. The water was filtered with a 0.02 um filter prior to adding the fiber. The final solution was not filtered. The particle size was measured with a Dynapro machine and it was assumed that the particles would behave similar to a globular protein. The solvent for the analysis was water.

### EXAMPLES 3-5, 3-6, 3-7

The fluorescein of Examples 3-1, 3-2, and 3-3 respectively was replaced by atrazine and the procedures repeated.

### EXAMPLES 3-11, 3-12, 3-13

The material from Example 3-5 was used for three tests where the nanoformulation product was placed in a third solvent for 0, 24, and 96 hours respectively. Particle size was measured after the indicated times.

Referring now to FIGS. 6A and 6B, additional embodiments of the invention include a continuous surface having edges defining collection regions that can be circular (e.g. O-rings), oval, square, or rectangular. Circular regions are shown in the figure. These regions can be used to harvest fibers according to the aspect of the invention illustrated in Examples 3-1 through 3-7. FIG. 6A is a frontal view of a belt of that optionally dips into a container with a solvent. FIG. 6B illustrates a side view of the belt of collection regions and shows cams over which the belt runs. The nozzles 1 and 2 may be positioned as shown for some embodiments with one or more divider insulators as shown. Sprocket holes may optionally be used on the belt to move the belt over the cams having matching sprockets in a controlled fashion or the belts may be moved without sprockets. In FIGS. 6A and 6B for example, when one O-ring is filled with fiber the continuous band moves so that the next O-ring is positioned to accept fibers and nanoparticles from the spray nozzles. The fibers can then be harvested from the O-rings. In some embodiments, the continuous band of O-rings may enter a tray containing solvent from which the fibers and/or nanoparticles may be harvested.

Referring now to FIGS. 7A and 7B, additional embodiments of the invention include a set of continuous belts that provides edges separated by a space that can be used to harvest the fibers from the collection site between the edges according to another aspect of the invention. FIG. 7A is a frontal view of a set of belts that dips into an optional container with liquid. FIG. 7B is a side view of the belts and shows cams over which the belt runs. The nozzles 1 and 2 may be positioned as shown for some embodiments with one or more divider insulators as shown. Sprocket holes may optionally be used on the belts to move the belts over the cams having matching sprockets in a controlled fashion or the belts may be moved without sprockets. In FIGS. 7A and 7B for example, when one area of the belts is filled with fiber the continuous band moves so that the next open area is positioned to accept fibers and nanoparticles from the spray nozzles. The fibers can then be harvested from the belts. In some embodiments, the continuous belts may enter a tray containing solvent from which the fibers and/or nanoparticles may be harvested.

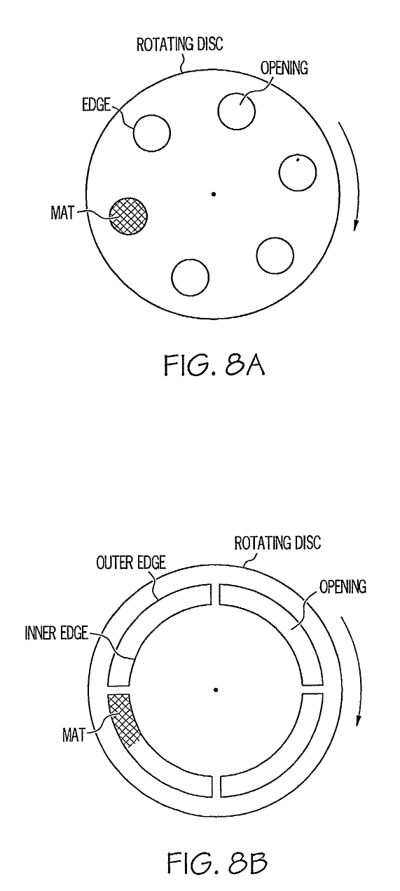

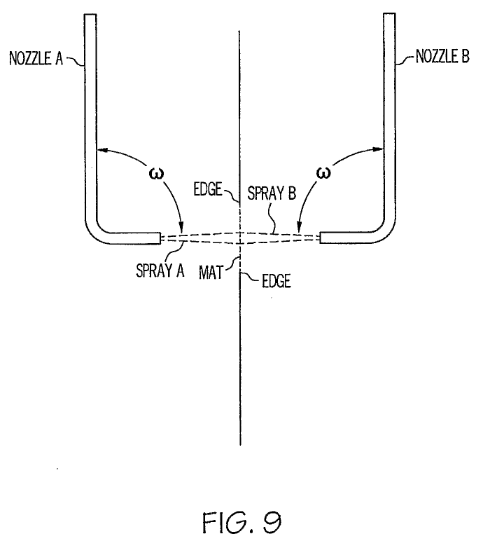

Referring now to FIG. 8A and FIG. 8B, the figures are schematic diagrams illustrating rotating discs 800, 850 respectively that can be used in the formation and collection of mats containing nanoparticles. FIG. 8A illustrates circular openings 810 that provide circular edges 820 for mat 830 formation. FIG. 8B illustrates semicircular openings 860 that are arc shaped. Mats 880 are built up between the inner edge 870 and outer edge 875 as shown. Nozzles configured as in the belt version shown in FIGS. 6A, 6B, 7A, and 7B, and also in FIG. 9 may be used with the discs.

Referring now to FIG. 9, the figure illustrates another embodiment of the invention that provides for an apparatus 900 having nozzles A and B 910 and 920 that are configured so as to spray toward each other. A collection area 930 between two edges 942, 944 may be used to build a mat 960 between two edges as shown. In some embodiments there may be not edges and the nozzles produce fibers, fibrils or particles. For head on spraying, angle ω is typically about 90°. However, for some embodiments Angle ω may be anywhere between about 30° and about 180°.

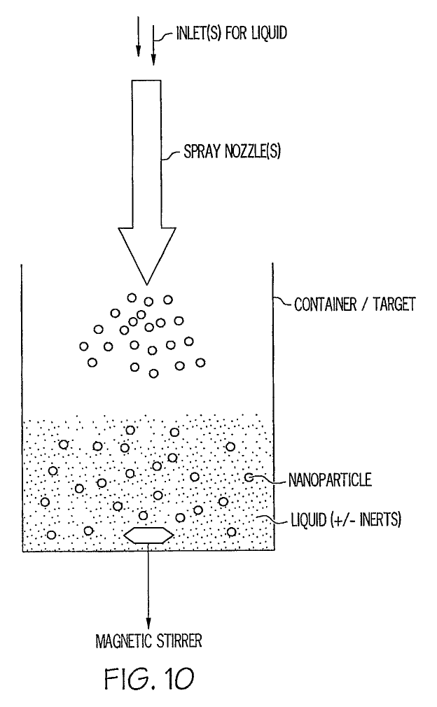

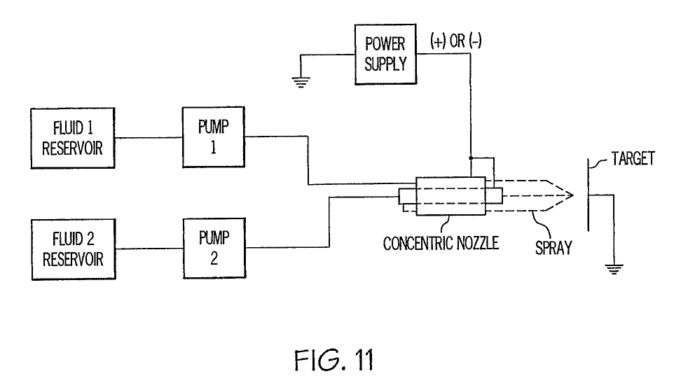

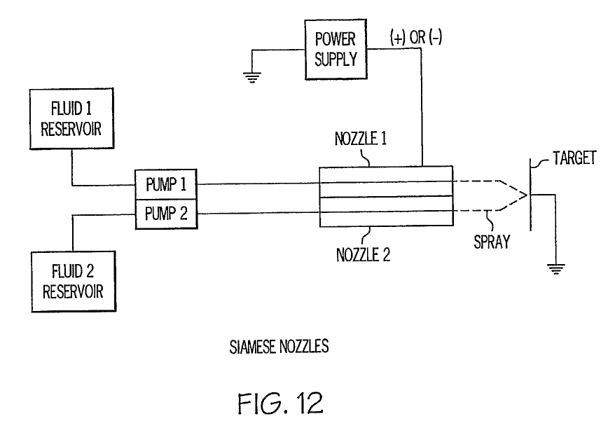

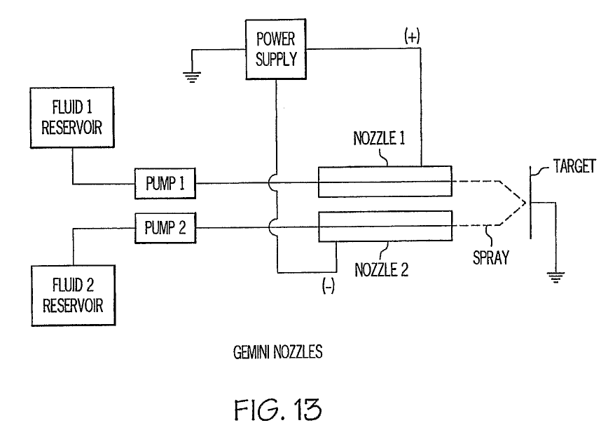

Referring now to FIG. 10, an additional embodiment of the invention provides for an apparatus 1000 for spraying nanoparticles using the spray techniques herein (e.g. ultrasonic atomization, spray drying, and spray freeze drying) into a container that contains a liquid. Nozzles or inlets 1010, 1012 spray the liquids toward a liquid 1020. This method avoids the formation of fibers or mats. The container 1080 may or may not contain a device 1085 (e.g. magnetic stirrer) to agitate or mix the liquid either mechanically or chemically. The additional embodiment includes a spray of nanoparticles using similar technology (EFET) or other know technologies in the field (e.g. ultrasonic atomization, spray drying, spray freeze drying) in to a container that contains among other things, a liquid, polymers for coating, excipients and other inert material that make the nanoparticles stable and efficacious. This invention does not require the production of fibers or mats or tablets and allows for the production of a material that contains nanoparticles that are ready for the end user. The nanoparticles can be generated by pushing a liquid or fluid through a nozzle that by known means generates an (1) aerosol; (2) particles; (3) fog; (4) particles; (5) gel; (6) semi-solid material; (7) capsules or (8) liquid spray. Once the resultant spray is sprayed into a container that may preferably contain a stirring or agitating mechanism, the particles solidify or gel into coated nanoparticles containing an active ingredient (e.g. agrochemicals, pharmaceuticals, fine chemicals, catalysts). The preferable size of these nanoparticles 1090 are in the 1-1000 nm range, and most preferably in the 10-500 nm range. Typical excipients used in the field are viscosity enhancing agents, surfactants, dispersing agents, binding agents, stabilizers, polymers, encapsulating agents, crystal growth inhibitors and the like. Typical stirring and agitating mechanisms include mechanical stirring, magnetic stirring, air bubbling, chemical agitation (e.g. use of effervescent agents) and the like. The final composition of the invention will include coated or uncoated nanoparticles in a liquid, semi solid or a solid media that is ready to use or can be worked up by the end user before application onto a target. The inlet to the nozzle that generates the nanoparticles can be one or more liquids that have either dissolved material or suspensions, emulsions or dispersions of the active ingredient. Additionally, the inlet can have more than one active ingredient and there can be more than one inlet.

In yet other embodiments of the invention, the apparatus provides for a partial vacuum in the area of the spray tip or tips. The sprayed materials are sprayed into a partial vacuum. The partial vacuum provides for enhanced solvent flashoff. This can enhance speed of the process, enhance flashoff of solvents that have higher boiling points or because of surface effects or otherwise are more difficult to remove. The partial vacuum may also enhance removal of certain excipients listed herein that are not needed or desired on or within the formed nanoparticles.

While the forms of the invention herein disclosed constitute presently preferred embodiments, many others are possible. It is not intended herein to mention all of the possible equivalent forms or ramifications of the invention. It is to be understood that the terms used herein are merely descriptive, rather than limiting, and that various changes may be made without departing from the spirit of the scope of the invention.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. A method of making a nanosuspension comprising:
a. providing nanoparticles at least partially encapsulated with an encapsulant;
b. providing a solvent in which the nanoparticles and the encapsulant is substantially insoluble, but causes the encapsulant to swell; and
c. mixing the at least partially encapsulated nanoparticles with the solvent to cause the encapsulant to swell.

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. A method of manufacturing a nanoparticle composition comprising:
preparing a first liquid formulation by combining an active ingredient and a first solvent;
preparing a second liquid formulation with a second solvent, wherein an optional encapsulant may be added; and
co-spraying the first liquid formulation and the second liquid formulation toward an edge to produce a nanoparticle composition at said edge.

47. The method according to claim 46, wherein the edge is the circular inner circumference of a collection region.

48. The method according to claim 46, wherein the edge comprises a plurality of collection regions disposed at a movable edge.

49. The method according to claim 46, wherein the edge comprises a set of two moving edges and the nanoparticle composition forms in the space between the two edges.

50. The method according to claim 49, wherein the set of two edges comprises two movable belts and the nanoparticle composition is formed as the belts move so as to form a continuous or semi-continuous mat between the edges.

51. The method according to claim 46, wherein the step of spraying is performed using one or more spray nozzles, movable with respect to the edge.

52. The method according to claim 46, wherein the edge is formed by a rotating plate having one or more openings that define an edge.

53. (canceled)

54. A method for making nanoparticle composition or suspension:
preparing a liquid formulation including an active ingredient and a liquid; and
spraying the liquid formulation toward the surface of a liquid, wherein the sprayed formulation is captured by the liquid without forming a mat and forms a nanoparticle composition or suspension of the active ingredient.

55. The method according to claim 54 wherein:
the step of spraying produces nanoparticles and the nanoparticles are captured by the liquid.

56. The method according to claim 55 wherein:
the step of preparing comprises preparing the liquid formulation by combining the active ingredient and a solvent.

57. The method according to claim 56 wherein
the step of spraying is performed by electrohydrodynamic spraying, electrostatic spraying, or ultrasonic atomization.

58. The method according to claim 54, further comprising:
establishing an electrical field between the liquid formulation and the surface of the liquid during spraying.

59. The method according to claim 58 further comprises:
performing the step of spraying in a partial vacuum.

60. The method according to claim 54, wherein the liquid into which the liquid formulation is sprayed includes an ingredient with which the liquid formulation reacts.

61. The method according to claim 54 wherein the nanoparticle comprises a catalyst.

62. The method according to claim 60, further comprising the step of
causing oligomerization or polymerization of at least one of the nanoparticle or an ingredient in the liquid into which the liquid formulation is sprayed.

63. The method according to claim 54 wherein the nanoparticle composition or suspension comprises excipients for the solvent to stabilize the nanoparticles, prevent crystal growth, Oswald ripening, or combinations thereof.

64. The method according to claim 54, further comprising:
preparing a second liquid; and
co-spraying the first liquid formulation and the second liquid formulation toward the surface of a liquid, wherein the sprayed formulations and combine and are captured by the liquid without forming a mat, and form a nanoparticle composition or suspension of the active ingredient.

65. The method according to claim 64, wherein:
the step of preparing a second liquid formulation comprises combining an active ingredient and a second solvent;
the step of co-spraying includes producing nanoparticles and the nanoparticles are captured by the liquid.

66. The method according to claim 64 wherein
the step of preparing a second liquid formulation comprises combining an encapsulant and a second solvent;
the step of co-spraying includes producing at least partially encapsulated nanoparticles and the at least partially encapsulated nanoparticles are captured by the liquid.

67. The method according to claim 66, further comprising the step of
exposing the at least partially encapsulated nanoparticle to a third liquid that causes oligomerization or polymerization to take place.

68. The method according to claim 66, further comprising the step of
exposing the at least partially encapsulated nanoparticle to a third solvent that causes the encapsulant to swell, and
causing the encapsulant to swell.

69. The method according to claim 66 further comprises:
performing the step of spraying in a partial vacuum.
